(19) United States
(12) Patent Application Publication
(10) Pub. No.: US 2006/0258985 A1
(43) Pub. Date: Nov. 16, 2006

(54) Graphical display of medication limits and delivery program

(76) Inventor: Claudia J. Russell, San Diego, CA (US)

Correspondence Address: FULWIDER PATTON, 6060 CENTER DRIVE, 10TH FLOOR, LOS ANGELES, CA, 90045, US

(21) Appl. No.: 11/127,841

(22) Filed: May 11, 2005

(51) Int. Cl. A61M 1/00

(52) U.S. Cl. 604/151; 604/891.1

(57) ABSTRACT

This invention describes a medication delivery system and method that displays in graphical form a selected delivery parameter or parameters in association with predetermined acceptable ranges of such values so that the clinician can visualize where in the acceptable range the selected value lies. The graphical form is a vertical bar graph in one embodiment that includes an acceptable area, a caution area, and an unacceptable area within which the selected delivery parameter value may fall. When the selected value falls in the caution or unacceptable areas, a text message is displayed cautioning against over dosage of the particular medical fluid being delivered and in another embodiment, an acceptable value of the delivery parameter is suggested. With such a system, any clinician monitoring the delivery of the medical fluid to the patient can immediately ascertain where in the care facility's acceptable range of delivery values this particular patient's medication is being delivered.

### BACKGROUND

The invention relates generally to the display of medical information in graphic form and more particularly, for graphically presenting medication delivery parameters including acceptable ranges of medication parameters and other data showing limits and selections.

Information intensive environments typically require observers of the information to make rapid yet correct decisions and this activity often results in higher levels of stress on those who must make decisions based on that information. An example of an information intensive environment can be found in the medical field where medication infusions must be given to patients. Infusions may be given in many different medicine environments, including an intensive care unit. In fact, intensive care units and other units are among the most information intensive environments in modem medicine. Monitors of increasing sophistication display vital functions. Information concerning modem medications, their uses, their dosages, and their possible interactions with other medications has become more available and the number of medications and information about those medications have also increased tremendously. Decisions in many medical environments are often critical and urgent while at the same time the workload of nurses and other clinicians is often quite high, which contribute to an increased stress level. These conflicting considerations, that is, increased information and reduced time to assimilate it, create critical junctures in patient care that may impair or delay the physician or other clinician from making accurate and safe judgments about the patient's immediate care.

The delivery of medications through infusion is an example where the need for fast and accurate decision making based on a great deal of information assaulting the clinician in a short time may arise. The enormous number of drugs available for infusion, the increase in knowledge as to possible interactions of those drugs, the need to determine the patient's status before infusing some but not all drugs may all arise as considerations prior to infusion. The need to convert units and many other information-based considerations may arise. Others, such as the effects of the patient's weight, age, size, body surface area, etc., must be considered in many, but not all cases. In some cases, they must be considered differently.

Most current information visualization techniques are less helpful in facilitating the ability to make a quick, yet correct and accurate decision. There have been few attempts to display this complex data graphically and in such a form as to assist the clinician in infusion delivery, intensive care units, and other high-stress and/or demanding environments in processing the data to derive quick and correct decisions effectively. Larger screens have been provided, color displays, larger sizes of text, and variations in text sizes have all been implemented over the years in attempts to achieve greater information transfer to the clinician; however, significant processing time is still required of the clinician to make a decision. Computer displays that mimic physical devices such as thermometers, gauges, and dials are readily associated with similar mental models. More complex information may require the use of already familiar or pictorial forms, appropriate emphasis, emergent features or combinations of these representations. Similarly, graphic representations of other shapes tend to communicate information more quickly. Yet rarely is this approach attempted.

The fluid flow rate or sequence of rates at which an infusion pump operates is typically selected based on a desired pattern of drug delivery appropriate to the specific circumstance. There are numerous factors that should be considered in the process of specifying a specific rate of fluid flow from a pump at a particular moment in time. Those factors may include: (i) the nature of the substance being infused, including the known pharmacokinetics and pharmacodynamics of a drug; (ii) the concentration or dilution of the substance in the fluid; (iii) the recipient, including sex, age, various measures of weight or size, the state of function of various organ systems, and ability to tolerate infusion of various diluent fluid volumes; (iv) the occurrence of change in measurable endpoints related to the actions or effects of the substance being infused; (v) the difference between the estimated concentration of the drug at the target site of drug action (as computed based upon actual measurements or based upon pharmacokinetic models and the prior history of the infusion and/or other factors), as compared to the desired concentration; (vi) local practices, policies, protocols, and regulations; or (vii) other considerations including operator judgement. The process by which a clinician takes the above considerations into account and chooses fluid flow rates for drug delivery from a pump is complex and if done incorrectly can lead to serious problems. The process is prone to error, especially because of the requirement for multiple computations and calculations. The complexities are aggravated by several factors. The staff training cost and the cost of preparing, updating, and distributing formularies, and the policy and procedure protocols within hospitals are large. The difficulty of establishing safe and uniform practices is compounded by significant local, regional, and nation-to-nation variations in practice patterns. The complexity of using such pumps may result in denial of needed therapy to patients because a care-giver lacks sufficient training, or does not have ready access or the time to review relevant knowledge or to use other tools (such as computational devices) to effect the process of delivering a needed medication infusion to a specific patient. It would be beneficial if a relative view of the delivery parameters programmed into the delivery device were available; that is, some comparison of the current parameters to an acceptable or predetermined range. This would communicate to the clinician the fact that the patient may receive a relatively high dose of a drug, a relatively low dose of a drug, or a dose falling within an acceptable range of doses.

Various technological approaches have been taken in the past to make infusion pumps more suitable for intravenous drug infusions. For example, companies have developed calculator-type infusion pumps that allow users to deliver drug diluted in a fluid by entering data such as drug concentration, patient weight, and desired doses and dose rates using dose delivery units such as mcg/kg or mcg/kg/min. Based upon these inputs, the pumps calculate the volumes and fluid flow rates to be delivered.

Some pumps include physical templates that can be attached to the pump and which electromagnetically modify (i.e., program) the infusion pump. These templates can be drug-, drug-concentration-, drug-container-size-, bolus-rate-, and dose-delivery-unit-specific. The templates contain knowledge that is useful for end users in that typical drug delivery doses and dose rates are printed on the physical templates. For each drug delivery configuration there must be a separate template. In addition, if a particular drug delivery configuration will be used simultaneously on multiple pumps within a facility, there must be available an equal number of identical templates, one for each pump. However, a pump resident template or drug library would overcome many of the foregoing problems. Many healthcare facilities prefer to have a standard drug list with standard delivery or dosing parameters. Innovation in this area has occurred and resident drug libraries are becoming more common in advanced infusion systems. For example, the Medley System from the ALARIS Products division of Cardinal Health, San Diego, Calif., U.S.A., includes modifiable drug libraries with which healthcare facilities may customize the dosing of each drug to achieve a more uniform approach and avoid serious delivery errors. Even though drug libraries have become available as resident within an infusion pump or pump system, the amount of information presented to a clinician by the pump can be large.

Hence a need has been recognized by those skilled in the art for an improved means of displaying information to busy clinicians so that safe medical services may be more efficiently provided to patients. A need has been recognized for presenting information in a more understandable and more rapidly comprehensible manner. There thus exists a long-felt but unfulfilled need in the medical art for methods and systems that will allow physicians or other clinicians to draw conclusions and make decisions as they are being bombarded by myriad forms of information in stressful environments. The present invention fulfills these needs and others.

### SUMMARY OF THE INVENTION

Briefly and in general terms, the present invention is directed to a system and method for providing data relevant to the operation of a medical device. The system and method of the present invention presents in easily comprehensible graphical form a medication parameter or parameters selected by the operator in relation to a predetermined parameter value range or ranges.

In one aspect, a system that graphically displays medication delivery information is provided. The system comprises an input device that produces a medical fluid signal representative of a medical fluid to be delivered, a medication delivery device that delivers the medical fluid, the medication delivery device operating in accordance with a selected value of a delivery parameter, a display for graphically presenting medication delivery information, a memory in which is stored a predetermined range of the delivery parameter associated with the medical fluid to be delivered, and a processor that receives the medical fluid signal, accesses the memory to retrieve the predetermined range of the delivery parameter associated with the medical fluid represented by the medical fluid signal, presents the predetermined range of the delivery parameter on the display in graphical form, and presents in graphical form on the display the selected value of the delivery parameter showing where in reference to the displayed graphical predetermined range the value of the delivery parameter is located, whereby a clinician can visualize where in the predetermined range the value of the delivery parameter lies.

In more detailed aspects, the graphical form of the displayed range of the medical fluid delivery parameter includes a plurality of numerals that provide values to the range. Also, the predetermined range of the delivery parameter is displayed in bar graph form, or other quickly recognizable graphical form. In a more detailed aspect, the bar graph may be in vertical or horizontal form. In another detail, the graphical form of the range of the medical fluid delivery parameter is displayed in summary form without the inclusion of numerals that would provide values to the range.

In other aspects, the predetermined range of a delivery parameter includes a maximum value and the processor displays a message when the selected value of the delivery parameter exceeds the maximum value. The message includes a graphical representation of the programmed dose setting with respect to the approved range of settings and includes a risk associated with high dose administration of the medical fluid. In another aspect, the message includes an alternative delivery parameter associated with the selected medical fluid. In another aspect, the predetermined range of a delivery parameter includes a minimum value and the processor displays a message when the value of the selected delivery parameter is less than the minimum value. The message includes a graphical representation of the programmed dose setting with respect to the approved range of settings. Also, the message includes a risk associated with a low dose administration of the medical fluid. In yet another aspect, the message includes an alternate delivery parameter associated with the selected medical fluid.

In other aspects, there is provided a system that graphically displays medication delivery information to a clinician, the system comprising an input device that produces a medical fluid signal representative of the identification of a medical fluid to be delivered, a pump that delivers the medical fluid, the pump operating in accordance with a selected value of a delivery parameter, a display, a memory in which is stored a library of identifications of medical fluids, each identification being associated with a predetermined range of the delivery parameter, and a processor that receives the medical fluid identification signal, accesses the memory to retrieve the predetermined range of the delivery parameter associated with the medical fluid identification signal, presents the predetermined range of the delivery parameter on the display in graphical form, and presents in graphical form on the display the selected value of the delivery parameter showing where in reference to the displayed graphical predetermined range the value of the delivery parameter is located.

In a further aspect, the memory in which is stored the library of identifications is located within the pump. In another aspect, the memory in which is stored the library of identifications is located remotely from the pump. In yet another aspect, the memory in which is stored the library of identifications is located in a controller disposed in communication with the pump.

Other aspects include that the predetermined range of the delivery parameter is displayed with a plurality of numerals indicating values on the displayed graphic range, the predetermined range of the delivery parameter is displayed in bar graph form. In more detail, the predetermined range is displayed in horizontal or vertical bar graph form or other quickly recognizable graphical form. In yet another aspect, the graphical form of the predetermined range of the medical fluid delivery parameter is displayed in summary form without the inclusion of numerals that would provide values to the range. The predetermined range of the delivery parameter includes a maximum value and the processor displays a message when the selected value of the delivery parameter exceeds the maximum.

In a further aspect in accordance with the invention, there is provided a system for medication administration management where the controller in communication with the pump is associated with a patient. Further, the controller is in communication with a network including access to the hospital pharmacy information system to obtain access to a complete listing of current prescriptions for the associated patient. The controller displays a list of medications ordered for the patient, the list including a graphical image of upcoming and overdue orders. The graphical reminder of upcoming and overdue orders can remain visible on other views presented by the controller to aid the clinician in rapidly selecting the medication that is due and provide the most efficient and effective care for the patient.

In a further aspect of the medication administration management system, the clinician can choose an ordered and due medication from the list provided and indicate that the dose was given. This indication can be communicated through the controller to the network and result in an update to the medication administration record.

In accordance with method aspects of the invention, there is provided a method of graphically displaying medication delivery information, the system comprising selecting a medical fluid to be delivered, selecting a value of a delivery parameter with which to deliver the medical fluid, storing a predetermined range of values of the delivery parameter associated with the medical fluid to be delivered, and graphically displaying the selected value of the delivery parameter in association with the stored predetermined range of values to graphically show where in the range the selected value falls. In more detailed aspects, the step of graphically displaying comprises displaying a plurality of value indicators in association with the graphical display of the predetermined range of values; the step of graphically displaying comprises displaying the predetermined range of values in horizontal or vertical bar graph form or other quickly recognizable graphical form. In another aspect, the step of graphically displaying comprises displaying the predetermined range of values in vertical or horizontal bar graph form or other quickly recognizable graphical form in summary without the inclusion of value indicators such as numerals that would provide actual absolute or relative values to the range.

In other detailed method aspects, the step of graphically displaying comprises displaying the predetermined range of values in a horizontal or vertical bar graph form or other quickly recognizable graphical form with a maximum value and displaying a message when the selected delivery parameter value exceeds the maximum value. Also, the step of displaying a message includes displaying a risk associated with high dose administration of the medical fluid, and the step of displaying a message includes displaying an alternative delivery parameter value associated with the selected medical fluid. In another aspect, the step of graphically displaying comprises displaying the predetermined range of values with a minimum value and displaying a message when the value of the selected delivery parameter is less than the minimum value. Also, the step of displaying a message includes displaying a risk associated with a low dose administration of the medical fluid. In yet another aspect, the step of displaying includes displaying an alternate delivery parameter associated with the selected medical fluid.

These and other aspects, features, and advantages of the present invention will become apparent from the following detailed description of the preferred embodiments which, taken in conjunction with the accompanying drawings, illustrate by way of example the principles of the invention.

### BRIEF DESCRIPTION OF THE DRAWINGS

FIG. 1 is a front view of a patient care system having a controller centrally located between two medication delivery devices, in this case two infusion pump devices mounted at either side of the controller;

FIG. 2 is a block diagram of components of the controller of FIG. 1;

FIG. 3 is a block diagram of the preparation of a drug library and the communication of that library to a patient care system, as well as the input of pump configuration and operation parameters and showing connection of the patient care system to a healthcare facility network, patient medication administration record (“MAR”), and pharmacy information system;

FIG. 4 is a diagram showing certain contents of a controller memory, including a drug library, an event log, and pump configurations including operating parameters;

FIG. 5 is a screen display in portrait orientation of a continuous infusion programming of Heparin showing the graphical display of a predetermined range for the dose of this drug with the patient weight selected to be 11 kg and concentration of the Heparin selected to be 10 unit/mL;

FIG. 6 is the screen display of FIG. 5 showing that the dose has been programmed to be 18.18 unit/kg/h and the graphical display of that selected dose in relation to the predetermined range for the dose of this drug shown in horizontal bar graph form;

FIG. 7 is a screen display in landscape orientation of a continuous dose or Dopamine with the concentration and VTBI already specified showing an entry of the patient weight of 70 kg and the relation of that selected weight to a predetermined range of weight for patients for this medicine at this concentration for this ward shown in graphical form of a vertical bar graph;

FIG. 8 is the screen display of FIG. 7 showing the landscape orientation of a continuous dose of Dopamine with the concentration, VTBI, patient weight, rate, and dose calculated or selected and showing in vertical bar graph form predetermined ranges of the dose and the rate and the relation to the selected dose and rate to those predetermined ranges, and further showing the presentation of a note relevant to this infusion and these selections;

FIG. 9 presents a summary graphical view of the dose of FIGS. 7 and 8 in which the predetermined range does not include numerals to give absolute valve to the range, but instead simply shows where in the predetermined acceptable range established by the healthcare facility this dose lies;

FIG. 10 is the controller of FIG. 1 showing the status of four channel, all of which are infusing medicines to a patient and showing the selected doses in graphical horizontal bar graph form, and further in the case of channel D, showing that the dose exceeds the predetermined acceptable range of the healthcare facility, further showing the remaining VTBI for each channel and showing an icon indicating that further infusions or medicine delivery or deliveries for this patient have been ordered;

FIG. 11 presents a representative “gas gauge” type of graphical format for communicating information to a clinician; and

FIG. 12 presents a representative “temperature gauge” type of graphical format for communicating information to a clinician.

### DETAILED DESCRIPTION OF PREFERRED EMBODIMENTS

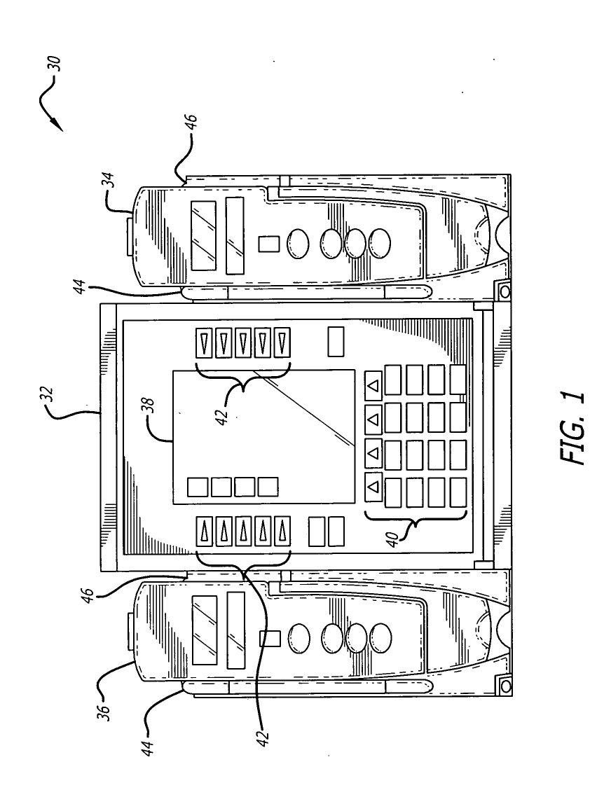

Referring now to the drawings in more detail in which like reference numerals refer to like or corresponding elements among the views, there is shown in FIG. 1 a modular, programmable patient care system 30. According to a preferred embodiment of the invention, this system comprises a controller 32 and at least one functional unit or medical instrument 34. In the embodiment shown, two functional units are mounted to the controller. These are a first infusion pump 34 and a second infusion pump 36. The controller performs four general functions in such a patient care system: 1) it provides a physical attachment of the system to structures such as IV poles and bed rails; 2) it provides power to the system; 3) it provides an interface between the system and external devices; and 4) except for certain specific information, it provides a majority of the user interface of the system. The controller contains an information display 38, which may be any type of display such as a liquid crystal display. The display may be used during setup and operating procedures to facilitate data entry and editing. The display may also be used to display various operating parameters such as volume to be infused (“VTBI”) for mounted individual functional units 34 and 36 and the current time of day, as well as other prompts, advisories, and alarm conditions. The controller also contains a plurality of hard keys 40 and soft keys 42 for entering data and commands. The numerical hard keys are used for entering numerical data, while the remainder of the hard keys, as well as the soft keys, are used for entering operational commands.

The soft keys 42 are arranged along the edges of the display 38 so as to interact with the display which will define the function of a particular soft key at any given time, as is well known to those of skill in the art. Therefore, a particular soft key when pressed will allow for the selection of an option, or an infusion or monitoring parameter, or other, which is displayed on the display adjacent the soft key. As noted, some hard keys 40 are also used for entering specific operational commands. For example, a hard key when pressed causes the system to change from standby to operating mode. As another example, a particular hard key or keys may be pressed to silence audio alarms and turn off electrical power to the controller 32.

The controller 32 also includes a tamper-resistant control function (not shown) which, when enabled, will lock out a predetermined set of controls. The controller preferably also contains at least one external communication interface which, in this embodiment, is located at the rear of the controller. The communication interface is preferably an industry standard RF communications card or a personal computer memory card international association (“PCMCIA”) slot for receiving PCMCIA cards, although one skilled in the art could select from a variety of commercially available communication means. Also located at the rear of the controller is at least one interface port such as an industry standard RS-232 port, although again, one skilled in the art could select from a variety of commercially available communication means. It is to be understood that although a preferred embodiment of the invention is described as containing an interface and at least one port, any number or combination of communication interfaces and ports could be included in the controller. It should also be understood that the embodiments of the present invention will be described in the context of a modular patient care system although those skilled in the art will recognize that the disclosed methods and structures are readily adaptable for broader application.

The interface and ports illustratively may be used to download drug libraries, drug delivery profiles, and other system configuration values, or may be used to upload event history data from the controller 32 or functional units 34 and 36. The interface and ports may also act as an interface to patient monitoring networks and nurse call systems or as an interface to external equipment such as barcode readers to provide means for inputting drug, patient, and other information from medication, patient records or other sources of information. Performing these functions with the ports and the interface will advantageously provide greater functionality and adaptability, cost savings, and a reduction in input errors. The ports and the interface may also be supplemented with a patient controlled analgesia (“PCA”) port (not shown) for use with a PCA functional unit such as that shown in U.S. Pat. No. 5,957,885 to Bollish entitled “Oximetry Monitored, Patient Controlled Analgesia System,” hereby incorporated by reference. The PCA port provides a connection to a remote hand-held “dose request” button that can be used by a patient to request a medication dose during PCA applications.

Located on both sides of the controller 32 are unit connectors 44 and 46 that are used to mount the functional units 34 and 36 to the controller 32. In this embodiment, these connectors provide physical support for the attached functional units and provide power and internal communication connections between the controller and the functional units. The functional units also contain these unit connectors 44 and 46 on either side so that other functional units may be connected to these functional units to thereby expand the patient care system in a side-by-side manner. A suitable unit connector is described in U.S. Pat. No. 5,601,445 to Schipper et al. entitled “Electrical And Structural Interconnector,” incorporated herein by reference.

The functional unit 34 or 36 may be selected from a wide variety of functional units including those for patient therapies and patient monitoring. More specifically as examples, the functional units may be an infusion pumping unit, a patient controlled analgesia (PCA) pump described above, a syringe pump, a pulse oximeter (see U.S. Pat. No. 5,957,885 to Bollish, cited above), an invasive or non-invasive blood pressure monitor, an electrocardiograph, a bar code reader, a printer, a temperature monitor, an RF telemetry link, a fluid warmer/IV pump, or a high rate IV pump (2000+ ml/hr), or other. It is to be understood that this list is for illustrative purposes only and that one skilled in the art could adapt functional units for other uses.

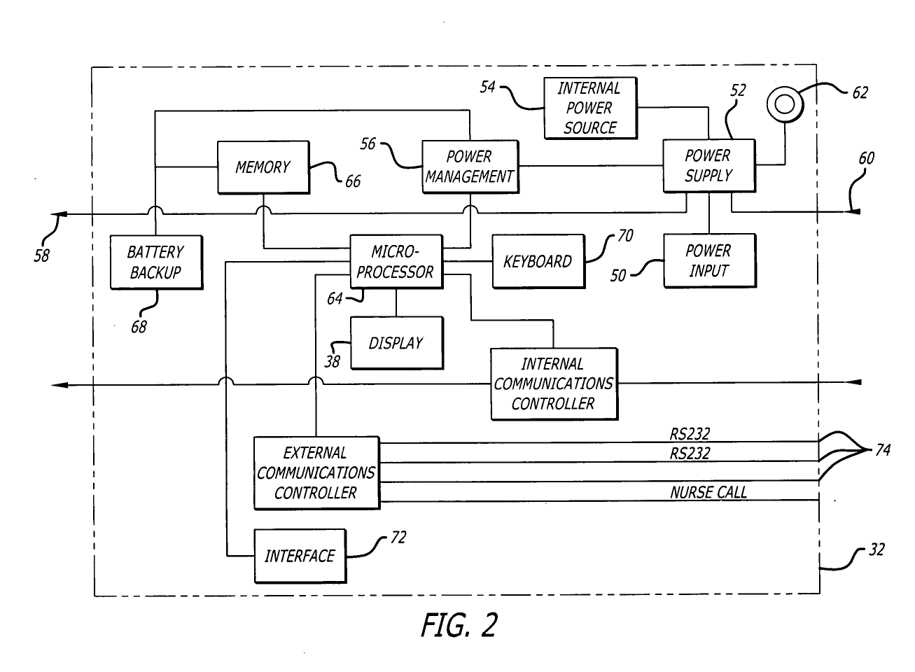

FIG. 2 is a schematic diagram for an embodiment of the controller 32. As shown, the controller contains a power input 50 for receiving power from an external power source and forwarding that power to a power supply 52. The controller also contains an internal power source 54 that may be used to maintain power to the system functions, including memory, when the controller is disconnected from an external power source. The power supply 52 converts power from either the external power input 50 or the internal power source 54 to voltages that are appropriate for operating all parts of the system. The power manager 56 controls the switchover between the two power sources, controls the charging of the internal power source 54, monitors the remaining capacity of the internal power source, monitors system power consumption under battery operation, and uses system power consumption and remaining battery capacity to estimate remaining system runtime on the internal power source. The power supply 52 also supplies power to the rest of the system through the power ports 58 and 60 as well as to the audio alarm 62, thereby enabling the audio functionality of the system.

The microprocessor 64 and the memory 66 receive and process data and commands from the operator, as well as communicate with and control the functional units 34 and 36 (FIG. 1) and other devices external to the system. It is to be understood that the memory, as well as other memories in the patient care system, may be any type of memory or any combination of memories that can be erased and reprogrammed without having to physically remove the memory from the system. Examples of such memories include, but are not limited to, battery-backed random access memory (RAM) and “flash” electronically erasable programmable read only memory (FLASH EEPROM). The battery backup 68 provides power to the memory to maintain the information stored in the memory in the event of loss of power from both the power input 50 and the internal power source 54. The controller 32 also contains a keyboard 70 (comprising the hard keys 40 and soft keys 42) and a display 38 as discussed in conjunction with FIG. 1. Further information concerning such a system can be found in U.S. Pat. No. 5,713,856 to Eggers, incorporated herein by reference.

In accordance with a preferred embodiment of the invention, the patient care system 30 as described may be programmed so as to provide a wide variety of functions and features to meet various user needs through the use of the interfaces of the controller 32. As discussed in conjunction with FIG. 1, the controller contains an interface 72 which is, in a preferred embodiment of the invention, a PCMCIA interface slot. The controller also contains ports 74 that in a preferred embodiment are industry standard RS-232 serial I/O ports. This interface and ports may be used to transfer drug libraries and drug delivery profiles into the system, load configuration values to the system, load new software or firmware to the system, and load event histories from the system. The interface and ports may also be used to control the system operation in certain situations, receive input from external devices such as bar code readers, and send current operating data to external devices such as monitoring systems. It is to be understood that although these exemplary uses of the interface and ports may be described above and below using one type of interface or port as an example, one skilled in the art will understand that many commercially available interfaces and ports could be used.

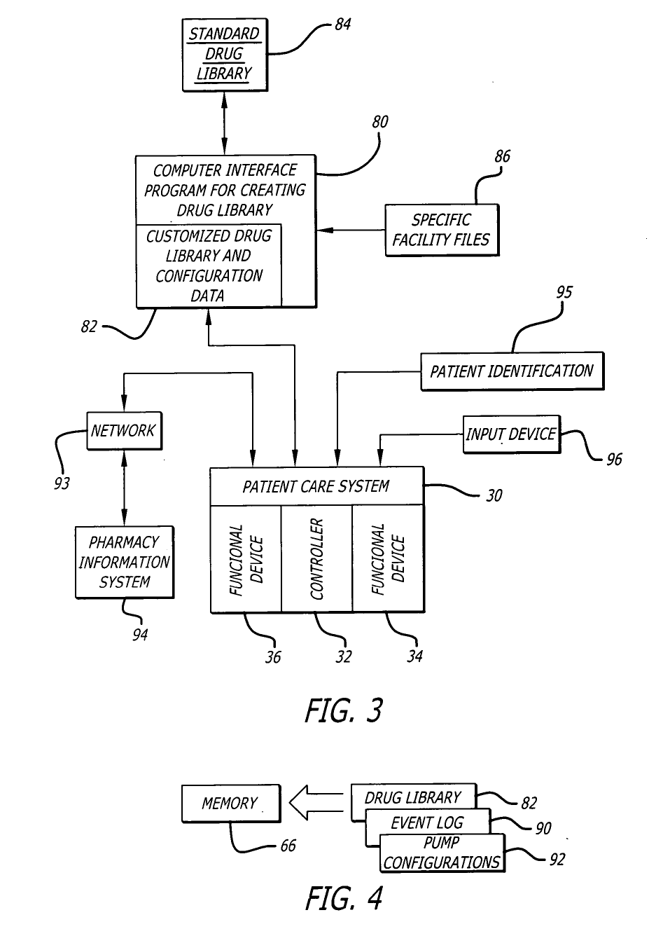

As mentioned, an interface or port may advantageously be used to download drug libraries to the patient care system 30. These drug libraries, which illustratively contain such information as the drug names, ranges of delivery parameter values such as proper concentrations, dosage units, and dose limits, and can be used to perform drug calculation-based infusions. Referring to FIG. 3, an external device 80, such as a personal computer, can be used to create drug libraries 82, which can be customized for each healthcare facility and each ward or practice group within the facility, such as ICU, neonatal, pediatric, OR, and others, and store these libraries on a PCMCIA memory card or communicate them directly to a patient care system 30. In the case of FIG. 3, direct communication is occurring.

The personal computer can also be used to prepare and load complex drug delivery profiles, or infusion protocols, to the patient care system 30. Various drug delivery profiles are known within the medical field. These profiles include multiple rate volume infusions, automated ramp up taper down infusions, multi-channel coordinated infusions, and multi-dose infusions. As is the case in the downloading of drug libraries 82, complex drug delivery profiles can be created and then stored on PCMCIA memory cards or loaded directly into the patient care system 30 as shown.

The customized drug library 82 may be created from a standard drug library 84 prepared by the healthcare facility in which a pump is located, or which may be a standard drug library used throughout the healthcare field. Additionally, specific healthcare facility files 86 may be considered in preparing the customized drug library for loading into the pump. These specific files may include non-standard limits for formulary drugs and drugs not in the standard formulary that are useful to a satellite facility, such as an oncology clinic for example.

Although PCMCIA cards are referenced above a number of times, it should be appreciated that other communication and storage media and devices may be used. For example, RF or Blue Tooth cards, and others, may be used for communication, and many other forms of data storage are possible. In addition to wireless communication techniques, wired communication may occur.

An interface or port may also be used to upgrade the patient care system with new software or firmware for new applications or to enhance performance. A specific example of this is when a new functional unit is added to the system that performs a function not previously available on the system. In this situation, a software domain corresponding to the new function must be downloaded to the controller 32 to which the new functional unit is mounted either directly or indirectly through mounting to another functional module or modules. The software domain corresponding to the new function allows the controller to understand and recognize the function of the functional unit and configure its user interface in a manner that permits a user to setup and perform the function.

An interface may also be used to upload event history to an external device. The controller 32 and each functional unit 34 and 36 within a patient care system has the capability of retaining information regarding its event history, including such information as infusion parameters, start time and end time of an infusion, incidents of alarms or advisories, and internal system errors. This event history illustratively may be used for updating patient records, troubleshooting, studying trends, and for other reasons. Event history data can be uploaded through and from the controller and each functional unit through RF communications, hard wiring or other means well know to those skilled in the art.

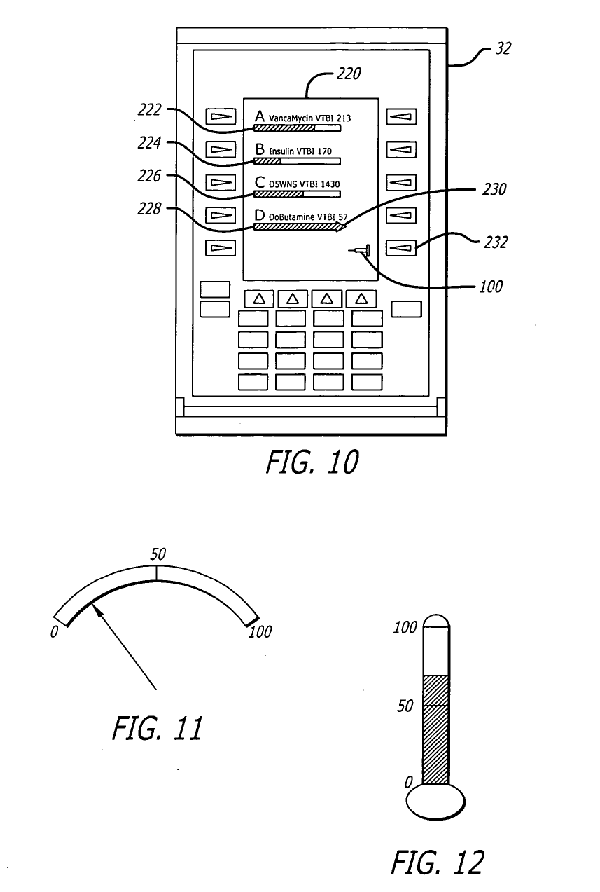

An interface of the patient care system 30 may also be used to connect to a network 93 to which the healthcare facility is also connected and which has access to the active patient medication orders available in the pharmacy information system 94. Since the controller 32 and the pump 34 can be associated with a patient 95, the network can query the pharmacy information system for a listing of current patient orders and send the data to the controller 32. The controller can display the information to the user in the form of a list on the display 38 of the controller or through other means. As is discussed below and shown in FIG. 10, a graphical element (icon) 100 can provide notice to the user that a dose is required or overdue. An input device 96, such as a keyboard, pointing device, or other device, can be used to select various data, such as a patient, medication, time frame, etc. When an infusion is programmed and started, a record is electronically made and sent to the network 93 to update the medication administration record of the patient. If an oral, topical, or injectable dose is presented on the list, the user may select that item and indicate whether the dose was administered. An electronic record of the administration of that dose is created and sent to the network to update the medication administration record.

Referring now to FIG. 4, the memory 66 of the controller 32 can contain the drug library or libraries 82, an event log or logs 90, and pump configuration settings 92, such as, but not limited to, profiles to be used in particular practice areas such as ICU, PED, etc., discussed above. Other data may also be stored.

As the controller 32 processes the data from the programming input such as the keyboard 96 or other input device, to the memory 66 and to the functional devices 34 and 36, it is preferable to input the information to the display 38 which will allow a clinician to review the information and to derive values therefrom in such ways so as to allow the clinician to make fast and efficient decisions about the patient by recognizing relationships between the data values.

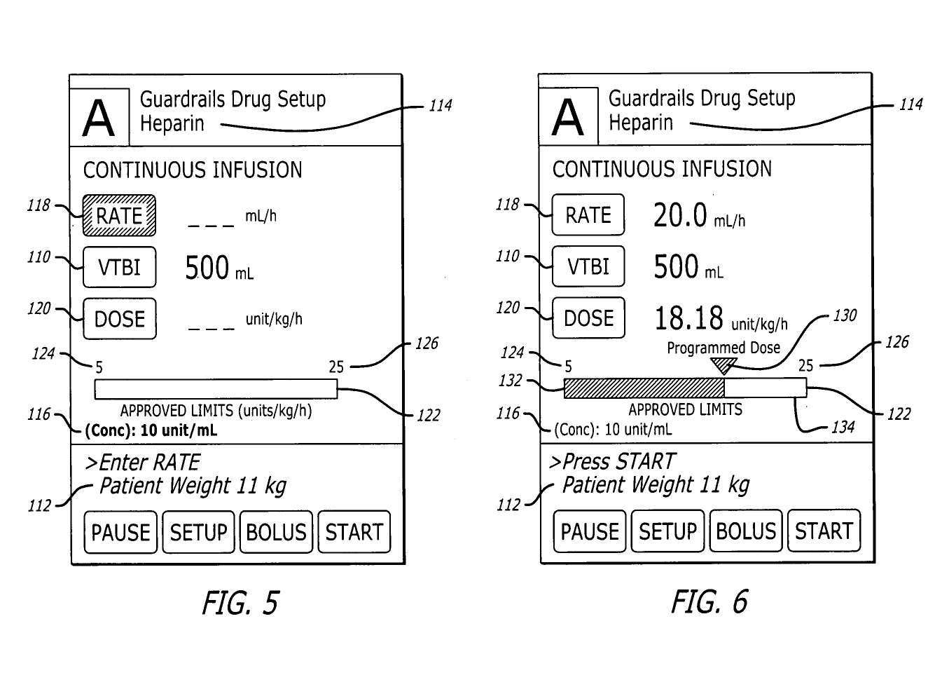

Referring now to FIGS. 5 through 10, various displays are shown for the efficient presentation of data to a clinician. The displays are in the context of an infusion pump; however this is for exemplary purposes only. Other instruments may incorporate aspects of the invention and present graphical displays to communicate data.

In FIG. 5, the programmed VTBI 110 of 500 mL is shown as is the programmed patient weight 112 of 11 kg that has been entered. The drug to be infused is labeled as Heparin 1 14 with a concentration (“conc”) 116 of 10 units/mL. A rate 118 and a dose 120 have not yet been selected. A horizontal bar graph 122 or chart is shown with the approved limits for this drug used in this area (pediatrics). In this case (pediatrics), the lower approved limit 124 is 5 unit/kg/h and the upper approved limit 126 is 25 unit/kg/h. Because no dose has been programmed into the unit, the horizontal bar graph of FIG. 5 is blank.

The same concentration 116, patient weight 112, and drug 114 data of FIG. 5 are shown in FIG. 6 along with the horizontal bar graph 122. However a dose 120 has been selected (18.18 unit/kg/h) and its relative position is shown in regard to the approved limits 124 and 126. In this particular embodiment, an equilateral triangle 130 having the words “Programmed Dose” above it is used. Also in this embodiment, a second indicator of the relative position of the selected dose in relation to the approved limits is used. The horizontal bar graph 122 includes a shaded, blackened, or different color segment 132 that indicates the selected dose (18.18 unit/kg/h) in relation to the outline or border 134 of the horizontal bar graph. Although not shown, two or more colors may be used in the horizontal bar graph. The selected dose indicated by the dose segment 132 may be a first color while the difference 136 between it and the maximum approved limit 126 may be a second color. In some cases, color has been found to be advantageous; however, the possibility of color-blind clinicians must be kept in mind when choosing the particular color or colors. It should also be noted that the display of FIGS. 5 and 6 is portrait in nature; however, other shapes may be used. Landscape or square or other shapes may be used to graphically display data.

As is well known by those skilled in the art, the clinician likely entered the rate in the example of FIGS. 5 and 6 and the dose was then calculated by the controller 32 (FIG. 1).

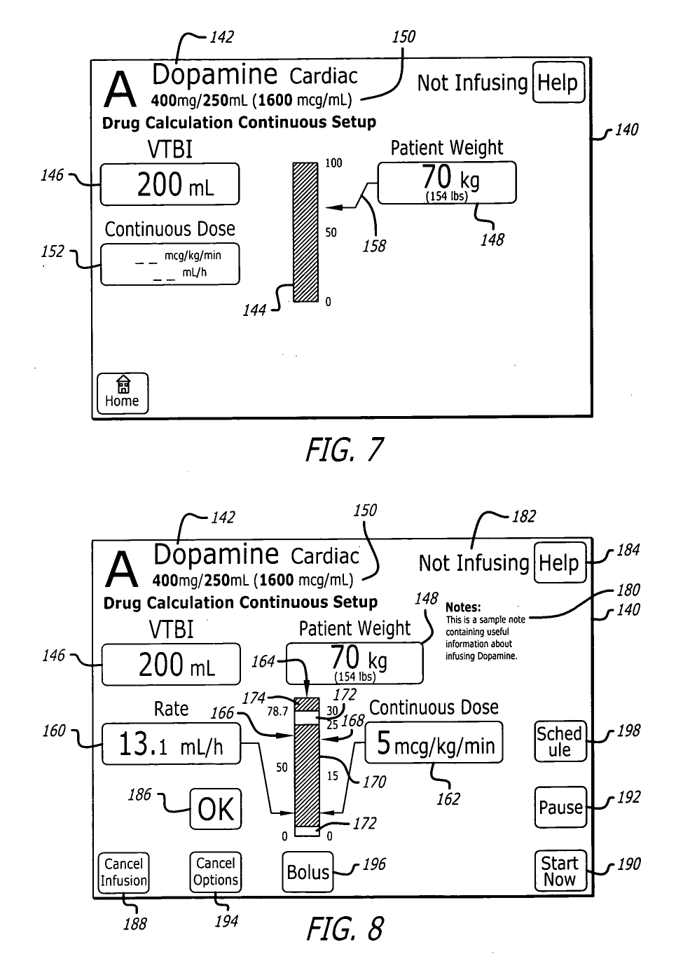

Referring now to FIG. 7, a landscape display 140 is presented in which a different drug, Dopamine 142, is being programmed for a different patient. A VTBI 146 of 200 mL, a patient weight 148 of 70 kg, and a concentration 150 of 400 mg/250 ml (1600 mcg/mL) have been programmed. No dose 152 has yet been programmed. Because this drug is weight limited, the vertical bar graph 144 is used to show the weight limits in easily readable graphical form. The minimum weight 154 is shown as zero kg while the maximum weight 156 is shown as 100 kg. This patient's weight of 70 kg is indicated with a lead line 158 from the weight box 148 which shows the weight in relative form to the acceptable limits of 0 to 100 kg. In this case, the vertical bar graph 144 is solid black, although it could be white or have color.

In accordance with another feature, the bar graph may be used to indicate the relative position of two drug infusion parameters in regard to approved limits at the same time. FIG. 8 presents the example of FIG. 7 with the further programming of a rate 160 and a dose 162. The vertical bar graph 164 in this case shows two scales of acceptable parameters; one 166 being the rate parameter and the second 168 being the dose parameter. Each scale has a different range. The range for the rate scale is from zero to 78.7 while the range for the dose scale is zero to 30. This vertical bar graph also contains a preferred area 170, two caution areas 172, and an unacceptable area 174. Two relative indicators 176 and 178 for each of the rate and dose respectively comprise a lead line and an arrowhead pointing at the vertical bar graph. In this case, segments of the vertical bar graph are blackened; however, color could be used. For example, the acceptable area may be the color green, the caution areas the color yellow and the unacceptable segment the color red. If a parameter, for example the dose, is programmed to the unacceptable segment 174, then a text message 180 under the heading of “Notes” would provide a message advising that there is a risk associated with high dose administration of this particular medical fluid. The message may take the form of “CAUTION, A DOSE THIS HIGH MAY CAUSE ARRHYTHMIA OR HEMODYNAMIC INSTABILITY.” Similarly, in this embodiment, if the programmed dose falls within the low caution area 172, a pertinent message 180 would be provided advising that there is a risk associated with under-dosing the drug. For example, the message may be “CAUTION, A DOSE THIS LOW MAY RESULT IN LITTLE OR NO THERAPEUTIC VALUE.” A glance at this vertical bar graph shows that in this case, both of the delivery parameters for rate and dose for this patient fall in the safe or “green” area. In this case, no text message would be provided, although in another embodiment, an informational message may be provided as desired.

Referring to FIG. 8 further, the status of the infusion pump 182 is stated near the upper right next to the soft button for requesting help 184. Once all appears to be accurate and acceptable, the “OK” key 186 may be pressed to accept the infusion parameters and to program the pump. Various additional keys exist such as a CANCEL INFUSION key 188, a START NOW key 190, and a PAUSE key 192. The functions of the foregoing are obvious to those skilled in the art. A CHANNEL OPTIONS key 194 is provided for selecting various options such as the air-in-line alarm setting, the contrast of the display, and other items. The BOLUS key 196 may be used to send a bolus of the drug to the patient, in the case where the drug being infused is one that can be safely administered as a bolus. Pressing the SCHEDULE key 198 instructs the pump to begin infusing at a scheduled time. Such a schedule would be previously programmed into the pump. The HELP key 184 permits the clinician to review information about the pump and pump programming as desired.

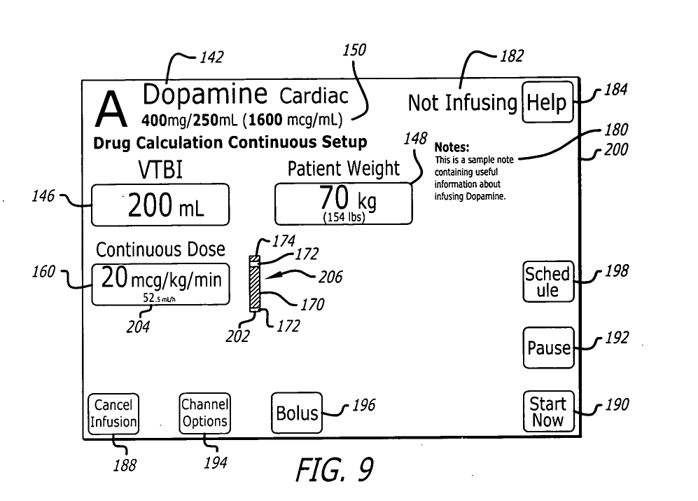

FIG. 9 presents an example of a graphical display 200 containing a vertical bar graph 202 showing a “summary” of the infusion parameter 204 adjacent to which the graph is located. It will be noted that the vertical bar graph is of much smaller size and is without value numerals. However, it does have the same acceptable 170, caution 172, and unacceptable segments 174 included in the vertical bar graph of FIG. 8. An arrowhead 206 is used to indicate where in the range the programmed parameter lies. In this case, the programmed dose is higher than that programmed in FIG. 8, as can easily and rapidly be seen from the summary vertical bar graph. It should be noted that the graphical graph could also take the form of a horizontal bar graph as well as other forms. Also, only one summary bar graph is shown and this pertains to just one programmed parameter. Other embodiments may include additional summary bar graphs positioned next to their respective alpha-numeric parameter. For example, a second summary vertical bar graph could be included in the display positioned next to the programmed rate, as well as yet a third bar graph positioned next to the patient weight 148. Other variations are also possible.

Referring now to FIG. 10, a display 220 on a controller 32 of a multi-channel pump system is shown in which all four channels (A through D) 222, 224, 226, and 228 respectively, are shown simultaneously as a summary graphical horizontal display. In each horizontal bar graph, the VTBI is indicated in numeric form, along with the name of the drug being infused, while the horizontal bar graph indicates the programmed dose in relation to the acceptable limits, without having numerals indicating those limits. The horizontal bar graphs are therefore summary in nature. The horizontal bar graph 228 for Dobutamine shows by means of a fully blackened bar graph and a rightward facing arrowhead 230 that the dose is higher than the maximum acceptable limit. Also as mentioned previously, a graphical element (an icon) 100, in this case a syringe, is in the lower right corner of the display and is used to indicate that there are scheduled medications due or overdue. This icon flashes to indicate doses that are past due. Pressing the soft key 232 next to the syringe icon will take the clinician to another screen upon which the due and/or overdue medications are listed. Other icons could be used in place of the syringe, such as an alarm clock. Additionally, the orientation of the syringe may indicate information. As an example, if the syringe were to point left, it may indicate an upcoming administration. If the syringe were pointing right, it may indicate a past due administration.

It should be noted that some screen displays appear in what is known as “landscape” format and some in portrait format; however, other forms are possible. For example, a square format may also be used. Some of the bar graphs are shown as having a “caution” range and an “unacceptable” range. However, in some cases, unacceptable ranges may not exist because the programming will not permit the entry of a rate outside an acceptable range. Therefore, a clinician could not even enter an “unacceptable” rate. However, a graphical presentation of the acceptable range would still be provided with an indication of the position in that range of the selected rate. Such a graphical display would be of value to the clinician since the relative position of the selected rate in the low-to-high range could be instantly seen. Some drugs can be more easily tolerated by patients while others are more difficult to tolerate. With the graphical display of the present invention, the clinician can instantly see if the drug under infusion is being infused at a rate that falls at the high end.

It should also be noted that other graphical representations of the information may be used. For example, the graphical formats in the drawings herein are horizontal or vertical bar graphs. However, a “gas gauge” format as shown in FIG. 11 may be used or even a “temperature gauge” format as shown in FIG. 12 may be used. Furthermore, color displays, grey scale displays, black and white displays and other types may be used as is found to be most appropriate.

Many sources of potential errors when prescribing and administering critical drugs to patients have been eliminated by means of dose error reduction software such as that made and distributed as the GUARDRAILS system by the ALARIS Products division of Cardinal Health, San Diego, Calif. Such software assists in catching errors before the dose was administered. The addition of the graphical displays disclosed and shown here will add a new layer of safety as they will aid in teaching clinicians the healthcare facility-generated appropriate ranges for drugs and will help normalize administration within those ranges. They further add the benefit of giving a quick reference to doctors making their rounds, nurses at shift change, administrators, and others involved with care of the patient when doses are due and whether an active infusion is being given above or below the approved limits. While an infusion outside the acceptable limits may be appropriate in certain circumstances, the graphical display in accordance with the invention herein that instantly shows the clinician that such an infusion is occurring acts as a reminder that this particular infusion and patient may need particular vigilance.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While variations have been described and shown, it is to be understood that these variations are merely exemplary of the present invention and are by no means meant to be limiting.

What is claimed is:

1. A system that graphically displays medication delivery information, the system comprising:
an input device that produces a medical fluid signal representative of a medical fluid to be delivered;
a medication delivery device that delivers the medical fluid, the medication delivery device operating in accordance with a selected value of a delivery parameter;
a display that graphically presents medication delivery information;
a memory in which is stored a predetermined range of the delivery parameter associated with the medical fluid to be delivered; and
a processor that receives the medical fluid signal, accesses the memory to retrieve the predetermined range of the delivery parameter associated with the medical fluid represented by the medical fluid signal, presents the predetermined range of the delivery parameter on the display in graphical form, and presents in graphical form on the display the selected value of the delivery parameter showing where in reference to the displayed graphical predetermined range the value of the delivery parameter is located;
whereby a clinician can visualize from observing the display where in the predetermined range the selected value of the delivery parameter lies.

2. The system of claim 1 wherein the graphical form of the displayed range of the medical fluid delivery parameter includes a plurality of numerals that provide values to the range.

3. The system of claim 1 wherein the predetermined range of the delivery parameter is displayed in horizontal bar graph form.

4. The system of claim 1 wherein the predetermined range of the delivery parameter is displayed in vertical bar graph form.

5. The system of claim 1 wherein the graphical form of the range of the medical fluid delivery parameter is displayed in summary form without the inclusion of numerals that would provide values to the range.

6. The system of claim 1 wherein:
the predetermined range of a delivery parameter includes a maximum value; and
the processor displays a message when the value of the selected delivery parameter exceeds the maximum value.

7. The system of claim 6 wherein the message includes a risk associated with high dose administration of the medical fluid.

8. The system of claim 6 wherein the message includes an alternative delivery parameter associated with the selected medical fluid.

9. The system of claim 6 wherein:
the predetermined range of a delivery parameter includes a minimum; and
the processor displays a message when the selected delivery parameter is less than the minimum.

10. The system of claim 9 wherein the message includes a risk associated with low dose administration of the medical fluid.

11. A system that graphically displays medication delivery information to a clinician, the system comprising:
an input device that produces a medical fluid signal representative of the identification of a medical fluid to be delivered;
a pump that delivers the medical fluid, the pump operating in accordance with a selected value of a delivery parameter;
a display;
a memory in which is stored a library of identifications of medical fluids, each identification being associated with a predetermined range of the delivery parameter; and
a processor that receives the medical fluid identification signal, accesses the memory to retrieve the predetermined range of the delivery parameter associated with the medical fluid identification signal, presents the predetermined range of the delivery parameter on the display in graphical form, and presents in graphical form on the display the value of the delivery parameter showing where in reference to the displayed graphical predetermined range the value of the delivery parameter is located;
whereby a clinician can visualize from observing the display where in the predetermined range the selected value of the delivery parameter lies.

12. The system of claim 1 I wherein the memory in which is stored the library of identifications is located within the pump.

13. The system of claim 11 wherein the memory in which is stored the library of identifications is located remotely from the pump.

14. The system of claim 11 wherein the memory in which is stored the library of identifications is located in a controller disposed in communication with the pump.

15. The system of claim 11 wherein the predetermined range of the delivery parameter is display with a plurality of numerals indicating values on the displayed graphic range.

16. The system of claim 11 wherein the predetermined range of the delivery parameter is displayed in bar graph form.

17. The system of claim 16 wherein the predetermined range is displayed in horizontal bar graph form.

18. The system of claim 16 wherein the predetermined range is displayed in vertical bar graph form.

19. The system of claim 11 wherein the graphical form of the predetermined range of the medical fluid delivery parameter is displayed in summary form without the inclusion of numerals that would provide values to the range.

20. The system of claim 11 wherein:
the predetermined range of the delivery parameter includes a maximum value; and
the processor displays a message when the selected value of the delivery parameter exceeds the maximum.

21. The system of claim 20 wherein the message includes a risk associated with high dose administration of the medical fluid.

22. The system of claim 20 wherein the message includes an alternative delivery parameter associated with the selected medical fluid.

23. A method of graphically displaying medication delivery information, the system comprising:
selecting a medical fluid to be delivered;
selecting a value of a delivery parameter with which to deliver the medical fluid;
storing a predetermined range of values of the delivery parameter associated with the medical fluid to be delivered; and
graphically displaying the selected value of the delivery parameter in association with the stored predetermined range of values to graphically show where in the range the selected value falls.

24. The method of claim 23 wherein the step of graphically displaying comprises displaying a plurality of numerals in association with the graphical display of the predetermined range of values.

25. The method of claim 23 wherein the step of graphically displaying comprises displaying the range of predetermined values in horizontal bar graph form.

26. The method of claim 23 wherein the step of graphically displaying comprises displaying the range of predetermined values in vertical bar graph form.

27. The method of claim 23 wherein the step of graphically displaying comprises displaying the range of predetermined values in vertical bar graph form in summary without the inclusion of numerals that would provide values to the range.

28. The method of claim 23 wherein:
the step of graphically displaying comprises displaying the range of predetermined values in vertical bar graph form with a maximum value; and
displaying a message when the selected delivery parameter value exceeds the maximum.

29. The method of claim 28 wherein the step of displaying a message includes displaying a risk associated with high dose administration of the medical fluid.

30. The method of claim 28 wherein the step of displaying a message includes displaying an alternative delivery parameter value associated with the selected medical fluid.
